(19) United States
(12) Patent Application Publication
(10) Pub. No.: US 2013/0096476 A1
(43) Pub. Date: Apr. 18, 2013

(54) APPARATUS AND METHOD FOR USING A CERVICAL TRACTION COLLAR

(71) Applicant: Richard J. Rogachevsky, Portland, OR (US)

(72) Inventor: Richard J. Rogachevsky, Portland, OR (US)

(21) Appl. No.: 13/515,251

(22) PCT Filed: Dec. 9, 2010
(86) PCT No.: PCT/US10/59727

§ 371 Date: Dec. 7, 2012

Related U.S. Application Data
(60) Provisional application No. 61/285,027, filed on Dec. 9, 2009.
(60) Provisional application No. 61/305,275, filed on Feb. 17, 2010.

(51) Int. Cl. A61F 5/055

(52) U.S. Cl. CPC A61F 5/055
USPC 602/18

(57) ABSTRACT

Cervical collars and methods are disclosed. In the various embodiments, an apparatus may include a cervical collar positionable on a neck of a user. A front portion may include at least one extensible chamber and an opposing rear portion having at least one extensible chamber. The front section and the rear section are mutually removably coupleable. A method in accordance with the various embodiments may include positioning the cervical collar about a neck of the user, and positioning a mouth piece in the user to engage the dentition of the user. Selected chambers in the cervical collar may be extended to urge the collar into contact with at least one of a chin and an occipital region of the user. The neck of the user may be tractioned using a selected mobilization pattern.

### PRIORITY CLAIM

The present application claims priority from U.S. Provisional Application Nos. 61/285,027, filed Dec. 9, 2009, and 61/305,275, filed Feb. 17, 2010, which applications are incorporated herein by reference.

### CROSS REFERENCE TO RELATED APPLICATIONS

This application is related to co-pending U.S. application Ser. No. 11/388,898, filed Mar. 24, 2006, and Ser. No. 11/388,336, filed Mar. 24, 2006, both of which claim priority from U.S. Provisional Application Nos. 60/737,833, filed Nov. 16, 2005, and 60/664,830, filed Mar. 24, 2005, all of which applications are incorporated herein by reference.

### BACKGROUND

Axial cervical traction devices may stretch the neck of a user in a selected direction. Many of these devices may traction approximately along a longitudinal axis of the user, while some may traction the neck of the user in slight flexion. Still others may traction the neck of the user in slight extension. It has been observed that these devices may cause discomfort to some users, and in some instances, provide only limited and temporary relief to the user. Although these devices may force decompression of spinal joint surfaces to some degree, other findings appear to indicate that they may not fully relax muscles in the user and therefore may not provide effective spinal decompression.

### BRIEF DESCRIPTION OF THE DRAWINGS

Various embodiments are described in detail in the discussion below and with reference to the following drawings.

FIG. 1 is an isometric view of a multi-directional cervical traction collar, according to the various embodiments.

FIG. 2 is a partial isometric view of a multi-directional cervical traction collar, according to the various embodiments.

FIG. 3 is an isometric view of the top front chamber of the multi-directional cervical traction collar, according to the various embodiments.

FIG. 4 is a side elevational view of the top front chamber of FIG. 3, according to the various embodiments.

FIG. 5 is an isometric view of one of the left rear top chamber and the right rear top chamber of the multi-directional cervical traction collar of FIG. 1, according to the various embodiments.

FIG. 6 is an isometric view of one of the left rear top chamber and the right rear top chamber of the multi-directional cervical traction collar of FIG. 1, according to the various embodiments.

FIG. 7 is a partial isometric view of the multi-directional cervical traction collar of FIG. 1, which will be used to describe a controller for controlling extension and/or retraction of the one or more chambers, according to the various embodiments.

FIG. 8 is an isometric view of a mouth piece, according to the various embodiments.

FIG. 9 is a flow chart describing a method of using a multi-directional cervical traction collar, according to the various embodiments.

FIG. 10 is a partial side elevational view of a mouth piece positioned in the mouth of the user, according to the various embodiments.

FIG. 11 is an isometric view of a head harness system, according to the various embodiments.

FIG. 12 is an isometric view of a head harness system, according to the various embodiments.

FIG. 13 is an isometric view of a head harness system, according to the various embodiments.

FIG. 14 is an isometric side view of the bottom chamber of the front portion, and the bottom chamber of the rear portion, according to the various embodiments.

FIG. 15 is a side elevational view of a rear chamber, according to the various embodiments.

### DETAILED DESCRIPTION

Mobilization of the neck, using non-axial, multi-directional traction devices may involve stretching the neck of the user on one side at a time, with simultaneous longitudinal traction. The longitudinal traction component may serve to decompress spinal joint surfaces in order to reduce joint pain and promote healing in the user. Non-axial, multi-directional cervical traction devices generally work with the body's neurological mechanism to help relax muscles by mobilizing and stretching opposite sides of the neck separately, which may include one or more of alternatingly stretching left and right portion of the neck, alternatingly stretching front and back portions of the neck, alternatingly stretching any one or more pairs of diametrically opposed portions of the neck that may lay between a front-to-back sagittal plane of the body and a side-to-side coronal plane of the body, and alternatingly stretching adjacent vertical portions of the neck to create circumduction of the neck. Accordingly, one portion of the neck may remain at rest and may be relaxed by an inhibitory reflex that may be initiated from an opposing side of the neck as it is stretched.

In addition, multiple directions of mobilization, as provided by these devices may also constitute an effective means to release scar tissue adhesions that may exist along planes of connective tissue fascia in the neck. The combined effect of muscle relaxation and connective tissue release, collectively referred to as soft-tissue release, may produce effective and potentially longer lasting decompression of spinal joints from the longitudinal traction. Soft tissue release may also help to decompress some spinal nerves, cranial nerves and peripheral nerves in the user. Soft tissue release may also help to decompress at least some vessels and channels in the user, which may result in an improved flow of blood and cerebral-spinal fluid, and improved drainage of lymphatic and venous return.

Accordingly, whereas axial cervical traction devices may be principally directed to the goal of producing temporary decompression of spinal joint surfaces, multi-directional cervical traction devices and methods may be configured to have a therapeutic effect on most aspects of the neuro-musculoskeletal system, including but not limited to joint surfaces, which may correspondingly help accelerate healing of body tissues and promote longer lasting relief from pain and various physical disabilities.

In the various embodiments, a multi-directional traction system may include a collar with one or more extensible chambers, a mouth guard, and a manually-operated or automated means for controlling extension of the chambers, which may include a source of a pressurized fluid medium coupled to the chambers that may be used to extend the chambers. The system may also include suitable conduits that fluidly couple the pressurized source to the chambers, and valves to direct the fluid medium to the separate chambers so that they may be extended individually or in various combinations. Pressure release components may be included in the various embodiments that may be configured to release the pressurized fluid in the one or more of the chambers.

In accordance with the various embodiments, the multiple chambers may be configured to extend generally upwardly, in a cephalic direction. The extension of the various chambers may be used to mobilize the neck of the user along one of the sagittal plane of the body, the coronal plane of the body, or in any intervening plane that lies between the sagittal and coronal planes of the body. Extension of a chamber may also mobilize the neck of the user along a longitudinal vector, which may also be referred to as traction.

In accordance with the various embodiments, multi-directional cervical traction devices may vary in the number and arrangement of chambers, and may apply forces at different points on the underside of the chin and the back of the skull, in order to generate various multi-directional mobilization patterns of the neck. In the description that follows, reference may be made to positions such as front, rear, top and bottom, and left and right. It will be understood that the foregoing positions generally refer to locations on the collar as it is worn by the user. For example, a top front chamber may generally refer to a chamber that is oriented towards a frontal portion of the user, while a rear chamber may generally refer to a chamber that is opposite the top front chamber. Similarly, a top chamber may generally refer to a chamber that is proximate the head of the user, while a bottom chamber may be positioned opposite the top chamber. Although one function of the bottom chambers may be to provide a stabilizing platform for the mobilizing action of the top chambers on the head and neck, some embodiments may not have a bottom chamber, in which case the chambers may extend from an area on one of the underside of the head and chin to an area on one of the upper back, chest or shoulder. The number and arrangement of chambers in the various embodiments may include, for example, the following combinations: Front and rear chambers, top front and/or top rear and/or bottom chambers, front and/or left rear and/or right rear chambers, top front and/or top left rear and/or top right rear and/or bottom chambers, rear and/or left front and/or right ^{p,} front chambers, top rear and/or top left front and/or top right front and/or bottom chambers, left front and/or right front and/or left rear and/or right rear chambers, and top left front and/or top right front and/or top left rear and/or top right rear and/or bottom chambers. It is understood that there may be still other chambers present, and additional arrangements of the chambers may be possible. For example, any chamber in any of the above-mentioned arrangements may be further sub-divided into any number of sub-chambers. The various embodiments may further include a bottom chamber, or alternatively, a bottom chamber may not be present. The bottom chamber may provide added stability to the top chambers, and may further produce axial traction of the neck.

The mobilization patterns that may be performed with multi-directional cervical traction devices may include at least one of the following: Alternation of cervical flexion and extension, alternation of cervical left lateral flexion and right lateral flexion, alternation of a cervical motion along any one or more diametrically opposed vector pairs that may lay between the sagittal plane of the body along which flexion and extension occurs, and the coronal plane of the body along which left and right lateral flexion occurs, and clockwise or counterclockwise circumduction of the neck.

Depending on the number of top chambers that may be present in the various embodiments, different mobilization patterns may be performed concomitant with traction. For example, in an apparatus that has four top chambers, a left top front chamber, a right top front chamber, a left top rear chamber and a right top rear chamber may be present, which may perform one or more of the following mobilization patterns: Simultaneous extension of all of the top rear chambers to mobilize the neck into forward flexion, simultaneous extension of all of the top front chambers to mobilize the neck into backwards extension, simultaneous extension of the left top rear chamber and the left top front chamber to mobilize the neck into right lateral flexion, and simultaneous extension of the right top rear chamber and the right top front chamber to mobilize the neck into left lateral flexion.

In another example, and in accordance with the various embodiments, an apparatus having three top chambers may include a top front chamber, a left top rear chamber, and a right top rear chamber that may perform one or more of the following mobilization patterns: Simultaneous extension the left top rear chamber and the right top rear chamber to mobilize the neck into forward flexion, extension of the top front chamber to mobilize the neck into a backwards extension, extension of the left top rear chamber to mobilize the neck into a right lateral flexion and extension of the right top rear chamber to mobilize the neck into a left lateral flexion. Furthermore, and in accordance with the various embodiments, systems having three or more top chambers may produce circumduction of the neck concomitant with traction, which may be accomplished by alternatingly extending the collar in adjacent vectors of motion.

Mobilization of the neck along vectors that may lie between the sagittal plane of the body along which flexion and extension occurs, and the coronal plane of the body along which left and right lateral flexion occurs, may be accomplished by simultaneous extension of more than one of the top chambers at a time, wherein at least one of the foregoing chambers may be extending at one or more of a different rate, which may be either a constant or a variable rate, and a different acceleration, and a different deceleration than the other chambers. Mobilization of the neck along vectors that may lie between the sagittal plane of the body and the coronal plane of the body may also be accomplished by using, and also in accordance with the various embodiments, more than four top chambers. For example, each of the four above-mentioned top chambers may include a left top front chamber, a right top front chamber, a left top rear chamber and a right top rear chamber that may be further divided into more than one sub-chambers.

It has been observed that lifting both the chin and the back of the head of a user to traction the cervical spine may produce greater spinal joint space separation as compared to lifting only the back of the head, and it has also been observed that this may put pressure on the Temporomandibular Joint (TMJ) of the user, which may result in pain and dysfunction in the TMJ, as well as reactive cervical para-spinal muscle spasms that tend to interfere with the intended separation of spinal joint surfaces. As a result, multidirectional traction, which may involve lifting both the head and the chin, may include the use of a mouth piece configured to be positioned in the mouth, to engage the dentition of a user. The mouth piece may serve at least one of the following functions: Preventing compression of the TMJ, and/or to decompress the TMJ. In the various embodiments, the mouth piece may be configured to engage the anterior aspect of the maxillary and mandibular dentition. The various embodiments may therefore serve to gap and/or prevent compression of the TMJ, the teeth, and the tongue of the user during traction. The various embodiments may also be configured to engage the molars and pre-molars in the posterior aspect of the dentition. The various embodiments may therefore be configured to act as a fulcrum, which may use the traction force itself to leverage the TMJ and produce decompression of the TMJ concomitant with cervical traction.

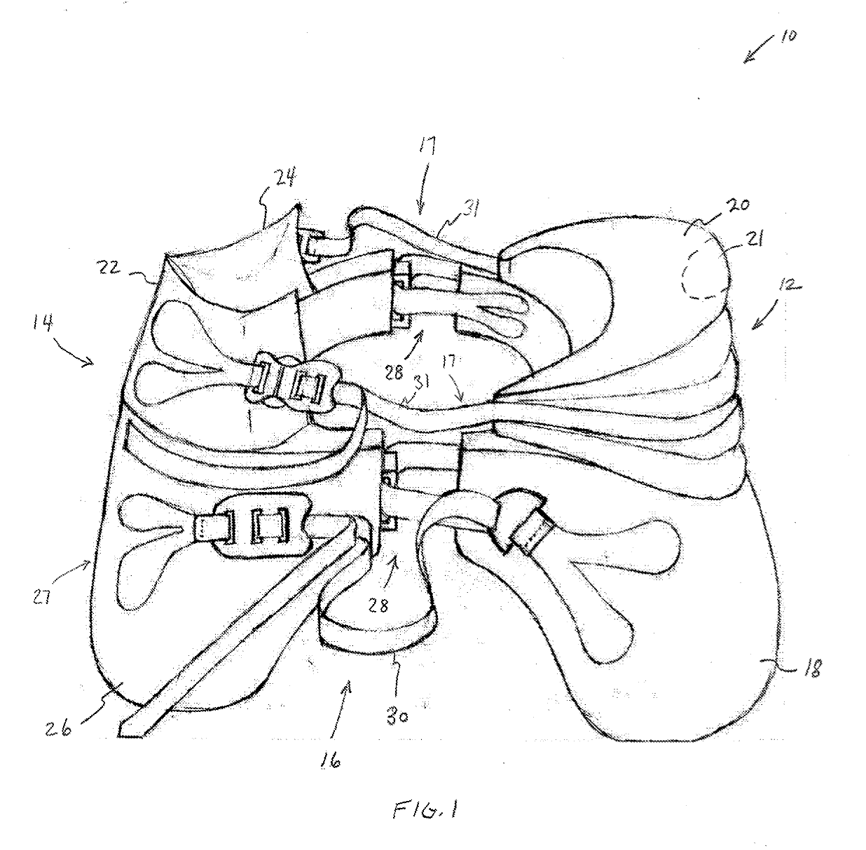

FIG. 1 is an isometric view of a multi-directional cervical traction collar 10 configured to perform multi-directional traction on a user, according to the various embodiments. The cervical collar 10 may generally include a front portion 12 and a rear portion 14 that may be coupled by a first strapping mechanism 16 and a second strapping mechanism 17. The first strapping mechanism 16 and the second strapping mechanism 17 include a first flexible member 30 and a second flexible member 31, respectively, which will be described in greater detail below. The collar 10 may generally include a plurality of chambers, which may include a bottom chamber 18 positioned in the front portion 12, which may further include more than one separately-extensible section, and one or more top front chambers 20 positioned in the front portion 12. The rear portion 14 may include one or more rear top chambers, such as a right rear top chamber 22 and a left rear top chamber 24. The rear portion 14 may also include one or more rear bottom chambers.

Each of the plurality of chambers may include an inlet (not shown in FIG. 1) that may be fluidly coupled to the chambers that may be coupled to one or more lengths of tubing (also not shown in FIG. 1) that fluidly couple the inlets (and therefore the chambers) to a fluid medium such as a gas, fluid or gel from a pressurized source (also not shown in FIG. 1). Accordingly, the pressurized fluid medium may inflate, extend, and cause a cephalic extension of each chamber. One or more shunting valves (also not shown in FIG. 1) may be configured to alternatingly distribute the gas, liquid or gel medium from the pressurized source to an extensible cavity within each chamber. Accordingly, one or more of the chambers may be extended simultaneously.

In the various embodiments, the pressurized source may include at least one of: One or more hand held inflation bulbs configured to deliver the pressurized fluid medium to a selected chamber by manually compressing the bulb; and/or one or more foot operated pedals configured to deliver the pressurized fluid medium to a selected chamber by depressing the pedal, and a motorized and automated mechanism configured to deliver the pressurized fluid medium to a selected chamber by motorized and automated means. One or more manually operated and/or automated pressure release features (not shown in FIG. 1) may also be included, as described in further detail below. In general terms, and as used herein, the term ‘inflation’ may include at least one of expansion and cephalic extension of the chambers by transferring a fluid or even a gel to the chamber, while the term ‘deflation’ may include retraction of the chambers by transferring a fluid or a gel away from the chamber.

With continued reference to FIG. 1, the front portion 12 and the rear portion 14 of the multidirectional cervical traction collar 10 may be configured to be coupled by the first strapping mechanism 16. In general, and in accordance with the various embodiments, the first strapping mechanism 16 may be configured to allow the user to conveniently couple, fasten and remove the collar 10 from around the neck, and be configured to allow convenient adjustment of the collar 10 to accommodate the users' neck. One function of the bottom chambers may be to provide stability in all of the top chambers during the mobilization and traction procedures. The collar 10 may also include side openings 28, which may be positionable at the left and the right sides of the neck of the user. The first strapping mechanism 16 generally includes a plurality of adjustable and first flexible members 30 configured to extend across each of the side openings 28. The side openings 28 of the collar 10 may allow the front portion 12 and the rear portion 14 the collar 10 to be adjusted according to a distance between a front and a back of the user's neck. The side openings 28 may also provide a means to suitably center the one or more top front chambers on an underside of the chin of the user. The side openings 28 may also provide a means to suitably center the one or more rear chambers over a base of an occipital bone of the user. Although FIG. 1 shows two side openings 28, it is understood that in other embodiments, a single side opening 28 may be present.

A chin cup 21 may be coupled to the one or more top front chambers 20, which may be configured to contact the chin of the user. In some embodiments, the chin cup 21 may be permanently attached to the one or more top front chambers 20, while in other of the various embodiments, the chin cup 21 may be removably coupled to the top front chambers 20 and detachable by releasable means, such as, for example, hook and loop fasteners. Accordingly, the detachable chin cup 21 may allow the user to select the use of a chin cup 21, or to select a chin cup 21 from a set that more closely conforms to the chin of the user. Alternatively, a chin cup 21 that is custom fit to the user may be used. In some embodiments, the chin cup 21 may include one or more layers of semi-rigid and flexible, resilient materials. For example, the chin cup 21 may include a first layer made of a semi-rigid material to provide structural support and a second relatively compliant layer made of an elastomeric material or a gel that serves to cushion the chin. In other embodiments, the first layer may contain raised areas designed to apply a pressure massage to the chin of the user, traction the soft tissues and drain lymphatic vessels that run along the underside of the chin and the anterior aspect of the neck. In embodiments with more than one top front chamber 20, the top front chambers 20 may be configured to be coupled along a portion or along substantially an entire length of their adjoining edges. In embodiments with more than one top front chamber 20, the chin cup 21 may have a flexible crease, or a hinge, that may overlay the gaps between the top front chambers 20, and allows independent extension of each of the top front chambers 20.

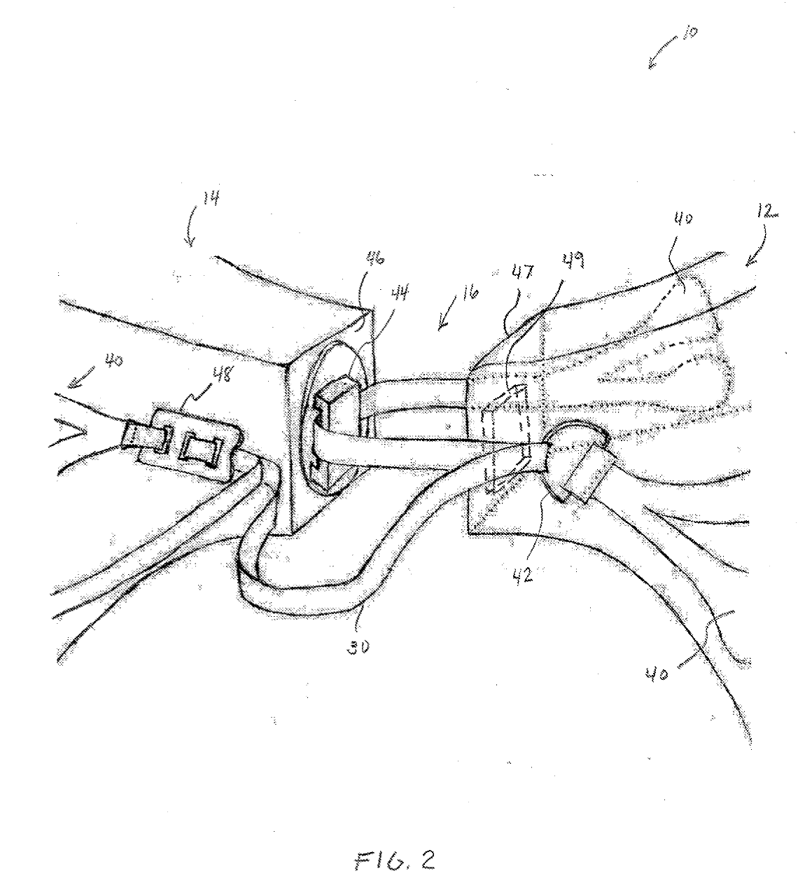

FIG. 2 is a partial isometric view of the multi-directional cervical traction collar 10 of FIG. 1, which will be used to describe the first strapping mechanism 16 in greater detail. In general terms, the first strapping mechanism 16 may be configured to couple the front portion 12 and the rear portion 14 the collar 10 by providing various anchoring points. For example, the front portion 12 and the rear portion 14 may include a plurality of attachment points 40 that may be adhesively bonded to the front portion 12 and the rear portion 14 the collar 10 using a suitable adhesive. Alternatively, other methods of attachment may be used, such as stitching, riveting, welding or still other methods of attachment. At least one of the attachment points 40 may include a D-ring 42 that may be configured to receive at least one of the first flexible members 30. For example, as shown in FIG. 1, the attachment point 40 coupled to the D-ring 42 may be attached to the front portion 12. A bracket 44 that is configured to receive the first flexible member 30 may be adhesively attached to an end portion 46 of the rear portion 14. An attachment point 40 that is suitably coupled to the rear portion 14 (e.g., by adhesive bonding, stitching, welding or riveting) may be coupled to a saddle lock 48 that is configured to receive the first flexible member 30, and to lockably retain the first flexible member 30 when the first flexible member 30 is suitably tensioned. The first flexible member 30 may be threadably routed from a selected one of the attachment points 40 and through the bracket 44. The first flexible member 30 may then be threadably received by the D-ring 42 and then routed to the saddle lock 48. Accordingly, when the first flexible member 30 is suitably tensioned, the front portion 12 and the rear portion 14 may be brought into contact, or approximated, at the end portion 46 of the rear portion 14, and an end portion 47 of the front portion 12. When a suitable tension is obtained in the first flexible member 30, the saddle lock 48 may arrest further movement of the first flexible member 30, so that the front portion 12 and the rear portion 14 may remain in contact, or at a set distance from each other. In order to enhance the stability of the front portion 12 and the rear portion 14 when the front portion 12 and the rear portion 14 are joined by the tensioned first flexible member 30, the end portion 47 may include a recess 49 that may be configured to receive the bracket 44 when the front portion 12 and the rear member 14 are joined or approximated. Although the attachment point 40 that is coupled to the D-ring 42 is shown attached to the front portion 12, it is understood that the D-ring 42 may be coupled to the rear portion 14, while the saddle lock 48 and the bracket 44 may be coupled to the front portion 12. For ease of securing and removing the collar 10 from the neck, quick release mechanisms may be associated with one or more of these fittings, including the saddle lock 48, the bracket 44 and the D-ring 42.

Returning briefly to FIG.1, and with continuing reference to FIG. 2, the top front chamber 20 may be configured to contact the underside of the chin, while the left rear top chamber 24 may be configured to contact the left side of the occiput, and the right rear top chamber 22 may be configured to contact the right side of the occiput. The left rear top chamber 24 and the right rear top chamber 22 may be configured to be joined to each other along a portion or along substantially an entire length of their adjoining medial edges. In the various embodiments, the second strapping mechanism 17 may extend continuously across the left side, the front, and the right side of the top front chamber 20, and may extend beyond the end portion 46 of the rear portion 14 to attachment points 40 coupled to the rear portion 14. A saddle lock 48 may also be provided, that is configured to threadably and lockably receive the second flexible member 31. In accordance with the various embodiments, the saddle lock 48 may be configured, for example, to include a clip that may be configured to releasably couple the front portion 12 and the rear portion 14 by lifting up on an end of the lock at a location where the flexible member 31 exits the saddle lock 48.

With continuing reference to FIG.1 and FIG. 2, the bottom chamber 18 of the front portion 12, the rear-bottom chamber 26 of the rear portion 14 may join at left and right sides of the neck to surround and enclose the neck of the user. Respective ends (e.g., end portions 46 and 47) of the front portion 12 and the rear portion 14 may be positioned proximate to the top of the shoulders of the user, while a portion of the front bottom chamber 18 may be positioned proximate to the chest of the user. A portion of the rear bottom chamber 26 of the rear portion 14 may be positioned proximate to an upper back of the user. In some embodiments, a second bracket 44 may be attached to the end 47 of the front section 18 to serve as an attachment or threading for the first flexible member 30.

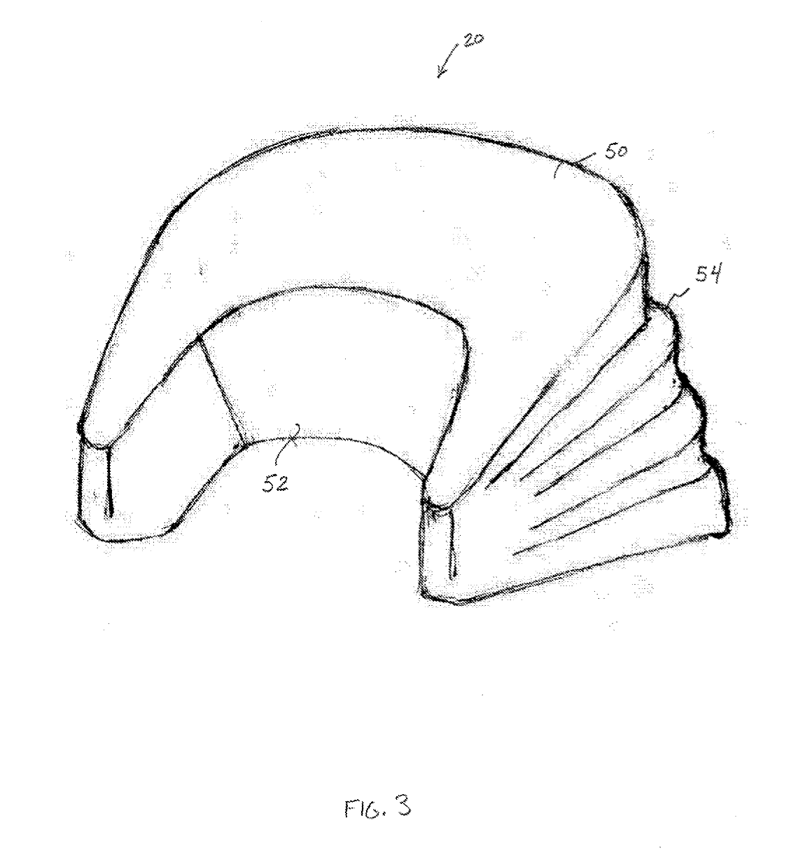

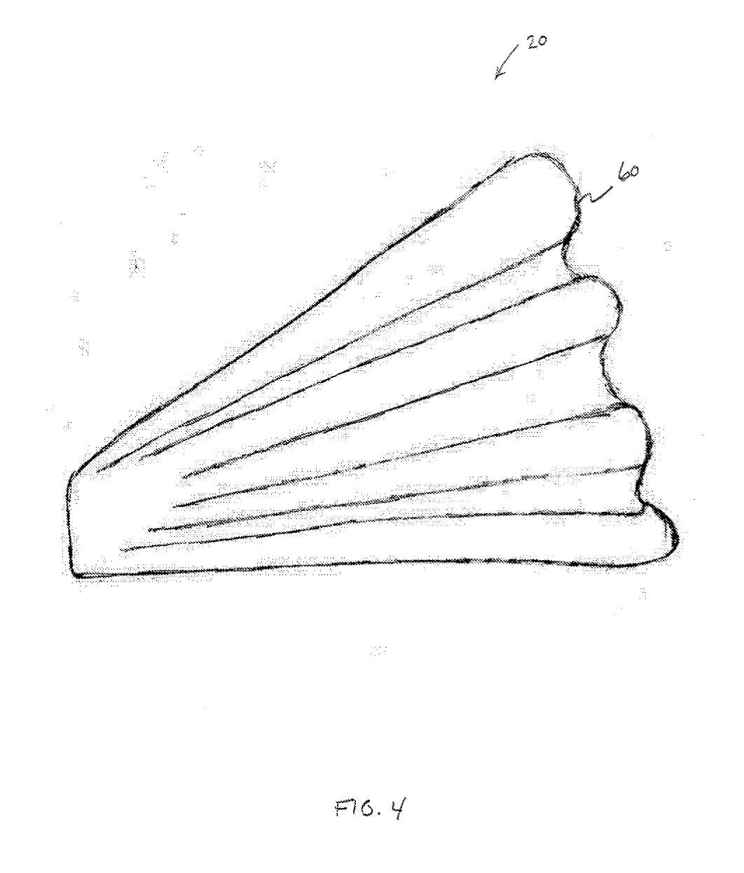

FIG. 3 is an isometric view of the top front chamber 20 of the multi-directional cervical traction collar 10 of FIG. 1, according to the various embodiments. The top front chamber 20 may include a contact surface 50 that may be suitably configured to receive the mandible and the chin of the user. The top front chamber 20 may also include a recess 52 that adjoins the contact surface 50. The recess 52 may be configured to receive the neck of the user. The top front chamber 20 may also include a plurality of antero-lateral corrugations 54 that may be configured to accommodate extension and retraction of the top front chamber 20. Referring now to FIG. 4, a side elevational view of the top front chamber 20 is shown. As shown therein, a plurality of undulations 60 may be formed in frontal and side surfaces of the top front chamber 20 that are configured to allow the top front chamber 20 to be readily extended and contracted. The undulations 60 may optionally include internal reinforcements (not shown in FIG. 4) that at least partially enforce a shape of the undulations 60 as they are extended and contracted.

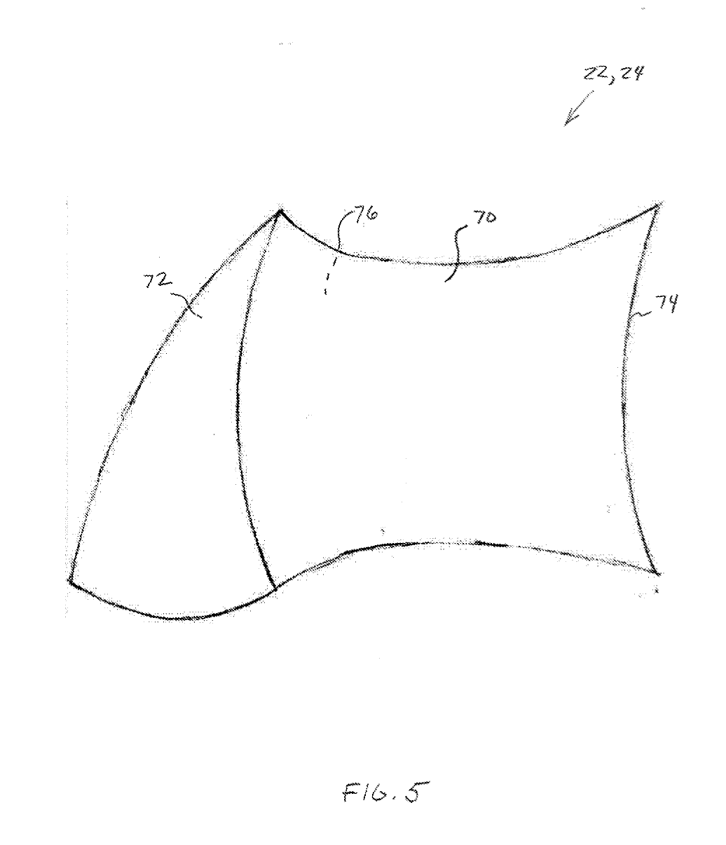

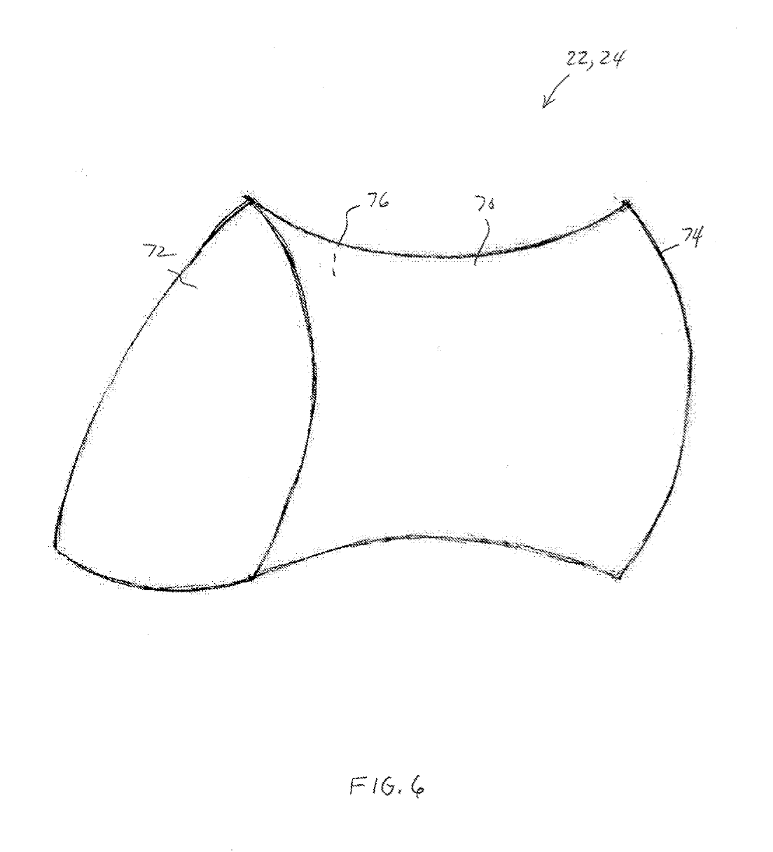

FIG. 5 is an isometric view of one of the left rear top chamber 24 and the right rear top chamber 22 of the multi-directional cervical traction collar 10 of FIG. 1, according to the various embodiments. The left rear top chamber 24 and the right rear top chamber 22 may include a contact area 70 configured to receive the occipital portion of the user's skull. The left rear top chamber 24 and the right rear top chamber 22 may also include a first side portion 72, an opposing second side portion 74 and a back portion 76. The contact area 70, the first side portion 72, second side portion 74 and the back portion 76 may be formed from resilient materials having different thicknesses to provide different expansion characteristics. Alternatively, the contact area 70, the first side portion 72, second side portion 74 and the back portion 76 may be formed from resilient materials having approximately similar thicknesses. Referring now to FIG. 6, one of the left rear top chamber 24 and the right rear top chamber 22 of the multi-directional cervical traction collar 10 of FIG. 1 is shown in an extended condition. Briefly, and in general terms, the left rear top chamber 24 and the right rear top chamber 22 may be extended to allow the contact area 70 to extend outwardly to contact the occipital portion of the head of the user, and support, and mobilize, the head of the user. While the left rear top chamber 24 and the right rear top chamber 22 are extended, the first side portion 72, second side portion 74 and the back portion 76 may be relatively non-extended.

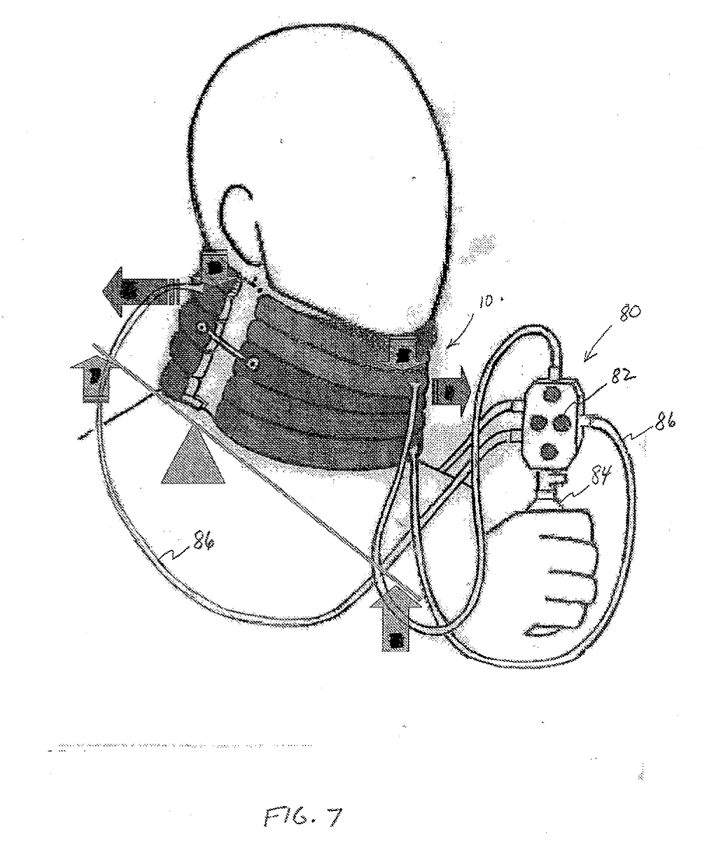

FIG. 7 is a partial isometric view of the multi-directional cervical traction collar 10 of FIG. 1, which will be used to describe a controller 80 for controlling extension and/or retraction of the one or more chambers, according to the various embodiments. The controller 80 may be configured to be operated by a hand of the user, and may include one or more manually-operable controls 82 configured to permit a pressurization fluid to be communicated from a supply 84 to the collar 10 through one or more fluid conduits 86 that may be fluidly coupled to the collar 10. Alternatively, and in another of the various embodiments, the controller 80 may include a foot-actuated apparatus, so that the manually-operable controls 82 may be operated by a foot of the user. In still others of the various embodiments, the controller 80 may be configured to extend and retract a first selected group of the chambers in the collar 10 using a hand-operated embodiment, as shown in FIG. 7, and to extend and retract a second selected group using a foot-operated embodiment of the controller 80. In still other of the various embodiments, the controller 80 may be a motorized controller, wherein the controls 82 may be at least partially assisted by an electromotive device. In still yet others of the various embodiments, the controller 80 may be at least partially automated, so that the expansion of the chambers in the collar 10 may be accomplished according to a set of programmed instructions.

With reference still to FIG. 7, the controls 82 configured to extend and retract the chambers may include a pressure release mechanism that may be configured to controllably regulate a pressure within the chambers of the collar 10 to a predetermined level. In accordance with the various embodiments, manually-operated pressure release mechanisms may include embodiments having a motorized pump that also may include at least one electrical switch to de-energize the motorized pump. In still other embodiments, the first strapping mechanism 16 or the second strapping mechanism 17 (FIG. 1) may have at least one clip that may be configured to release with the application of manual pressure, and which may immediately remove at least some pressure from all contact points on the head and mandible of the user. Other embodiments may include at least one manually operated valve coupled to the bottom chamber 18 that may be configured to retract one or more sections of the bottom chamber 18, which may at least partially releases pressure from one or more contact points on the head and mandible of the user. Still other embodiments may include at least one manually operated valve coupled to the one or more top front chambers 20, the right rear top chamber 22 and the left rear top chamber 24 configured to retract at least one of the top front chambers 20, the right rear top chamber 22 and the left rear top chamber 24, which may at least partially release pressure from one or more contact points on the head and mandible of the user.

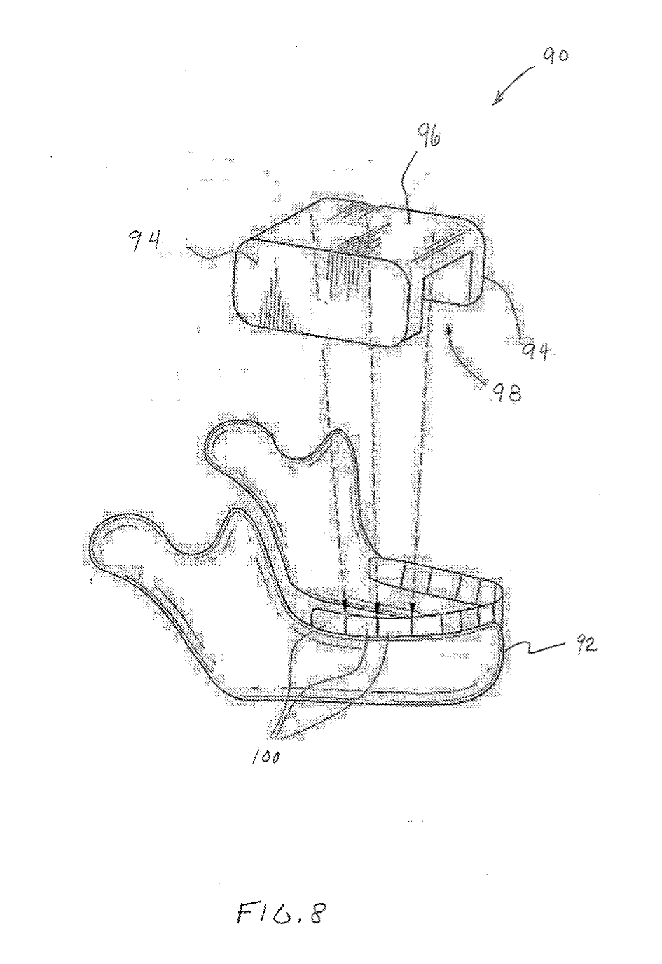

FIG. 8 is an isometric view of a mouth piece 90, according to the various embodiments. In general terms, the mouth piece 90 may be suitably configured to assist a temporomandibular joint of the user during the application of tractive forces imparted to the user by the multi-directional cervical traction collar 10 of FIG. 1. The mouth piece 90 may be configured to be grasped between a lower mandible 92 and an upper maxilla (not shown in FIG. 8). The mouth piece 90 may include opposing and spaced-apart side walls 94 and a top wall 96 that may extend between the side walls 92. Accordingly, the top wall 96 and the side walls 94 define a channel 98 that may be configured to receive one or more of the molars 100 in the lower mandible 92 of the user. The mouth piece 90 may be generally formed from any polymeric, resilient material so that the molars 100 and other soft tissues proximate to the molars 100 are not injured when the mouth piece 90 is grasped between the lower mandible 92 and the upper mandible (not shown).

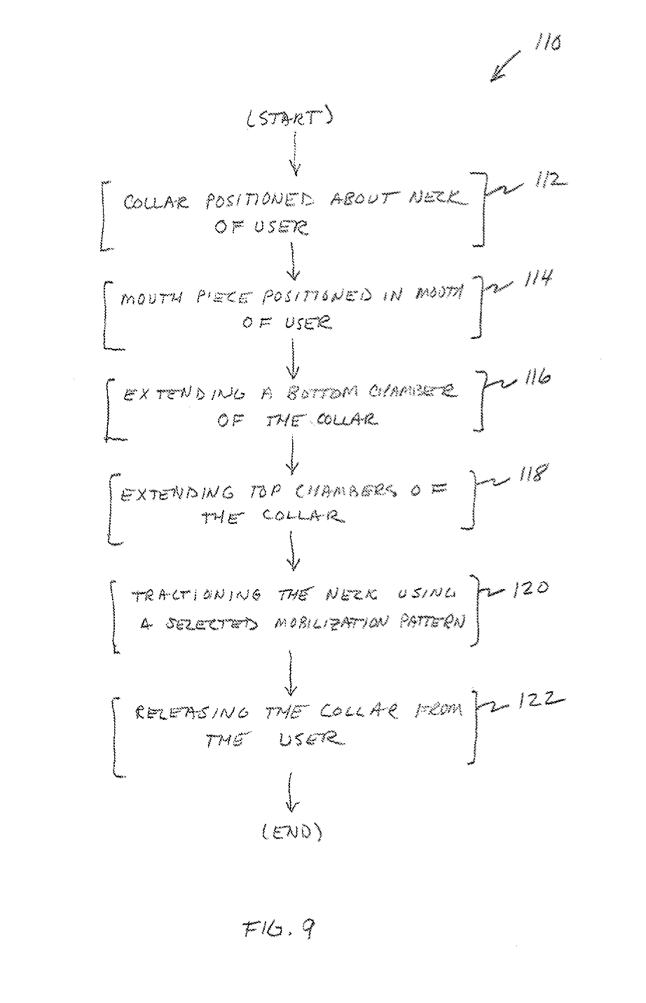

FIG. 9 is a flow chart that will be used to describe a method 110 of using a multi-directional cervical traction collar, according to the various embodiments. At 112, the collar 10 (FIG.1) may be positioned about the neck of the user. The collar 10 may then be adjusted about the neck by suitably adjusting the first strapping mechanism 16 and the second strapping mechanism 17. At 114, the mouth piece 90 (FIG. 8) may be positioned in the mouth of the user. In particular, the mouth piece 90 may be positioned on the lower mandible 92 of the user, so that it engages the dentition of the user. At 116, at least one of the bottom chamber 18 positioned in the front portion 12, and the right rear bottom chamber 26 positioned in the rear portion 14, may be at least partially extended so that the top front chamber 20 positioned in the front portion 12, and the right rear top chamber 22 and the left rear top chamber 24 positioned in the rear portion 14 are brought into contact with the chin portion and the occipital portion of the user, respectively. At 120, the neck of the user may be tractioned using a selected mobilization pattern, such as, for example, simultaneous extension of all of the bottom rear chambers to mobilize the neck into forward flexion, simultaneous extension of all of the front chambers (e.g., the bottom chamber 18 and at least one of the top front chambers 20) to mobilize the neck into backwards extension, simultaneous extension of the left rear top chamber 24 and one of the ipsilateral top front chambers 20 to mobilize the neck into a lateral flexion, and simultaneous extension of the right rear top chamber 22 and another of the ipsilateral top front chambers 20 to mobilize the neck into another lateral flexion. Still other mobilization patterns are possible, as described in detail above. At 122, the collar 10 may be released. In particular, the chambers (e.g., the front bottom chamber 18, the rear bottom chamber 26 the front top chambers 20, the left rear top chamber 24 and the right rear top chamber 22) may be deflated, either simultaneously, or sequentially, releasing pressure from all contact points on the head and the mandible. The first strapping mechanism 16 and the second strapping mechanism 17 may be released and the front portion 12 and the rear portion 14 may be removed from the user.

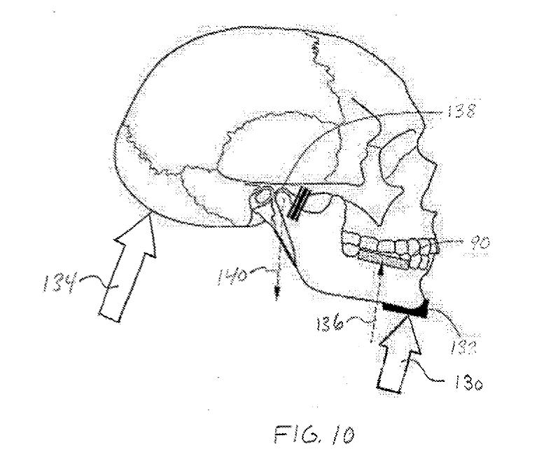

Turning now to FIG. 10, the function of the mouth piece 90 during execution of the method 110 will now be discussed in greater detail. With the mouth piece 90 positioned in the user's mouth, a first force vector 130 may be applied to a chin 132 of the user by extension of the bottom chamber 18 and/or the top front chambers 20. A second force vector 134 may also be applied to the occipital region of the user by extension of the rear bottom chambers 26 bilaterally, the left rear top chamber 24 and the right rear top chamber 22. Accordingly, the mouth piece 90 may establish a fulcrum point 136. The fulcrum point 136 accordingly allows the first force vector 130 and the second force vector 134 to be suitably balanced. A temporomandibular joint (TMJ) 138 in the user may therefore be unloaded, by moving a lower portion of the TMJ 138 downwardly in the direction 140.

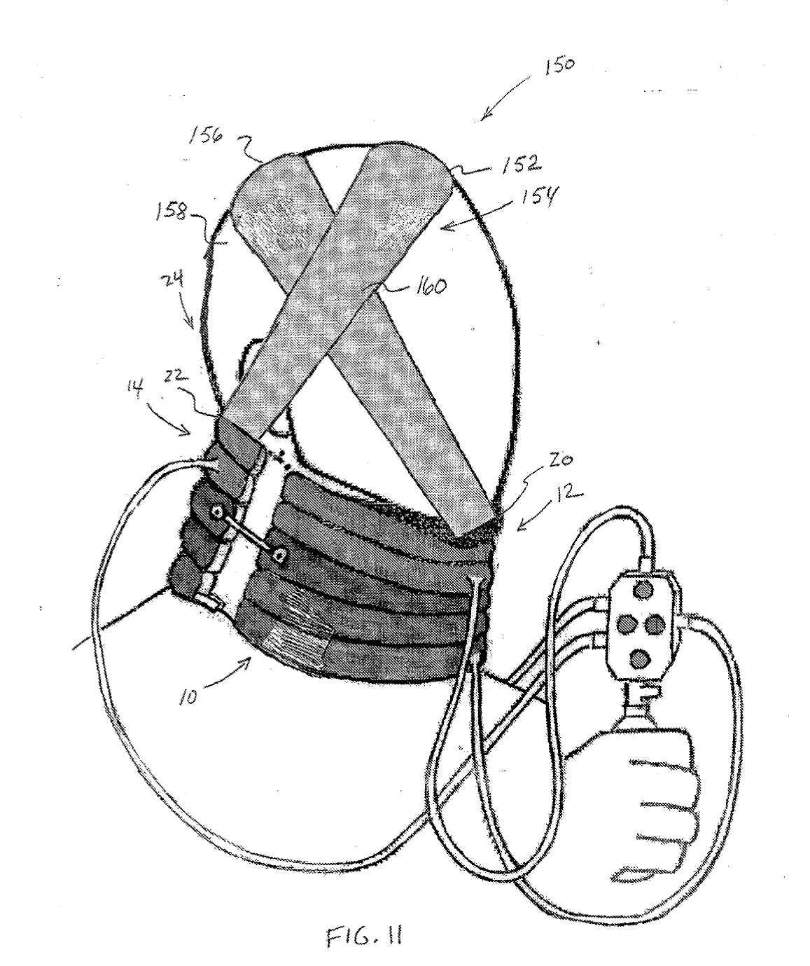

FIG. 11 is an isometric view of a head harness system 150, according to the various embodiments. The head harness system 150 may be coupled to the multi-directional cervical traction collar 10 of FIG. 1, or to other similar cervical traction devices, and may include a forehead strap 152 that may be configured to extend from a selected location of the rear portion 14, across a forehead 154 of the user, and extend to the same, or even another selected location on the rear portion 14. In the various embodiments, the forehead strap 152 may be coupled to the right rear top chamber 22 of the multi-directional cervical traction collar 10 at a first end, and to the left rear top chamber 24 at an opposing second end. The head harness system 150 may also include a crown strap 156 that may be configured to extend from one or more selected locations on the front portion 12, across a crown 158 of the user, and to the same, or another location on the front portion 12. In the various embodiments, the crown strap 156 may be coupled to the one or more top front chambers 20. The forehead strap 152 may be coupled to the selected locations of the rear portion 14 using adhesives, or using a variety of mechanical fasteners, such as releasable snaps, buttons, rivets, or even hook and loop fasteners, or by welding. Similarly, the crown strap 156 may be coupled to the selected locations on the front portion 12 using adhesives, or using a variety of mechanical fasteners, or by welding. The forehead strap 152 and the crown strap 156 may be formed from any flexible and resilient material, including various fabrics and polymers having elastomeric properties. In the various embodiments, the forehead strap 152 and the crown strap 156 may be mutually coupled at an intersection 160 of the forehead strap 152 and the crown strap 156. Alternatively, the forehead strap 152 and the crown strap 156 may be merely overlaid, and not otherwise coupled at the intersection 160. In other embodiments, the forehead strap 152 and the crown strap 156 may be integrally formed. In still other embodiments, the head harness system 150 main include two or more of the forehead straps 152, or alternatively, two or more of the crown straps 156.

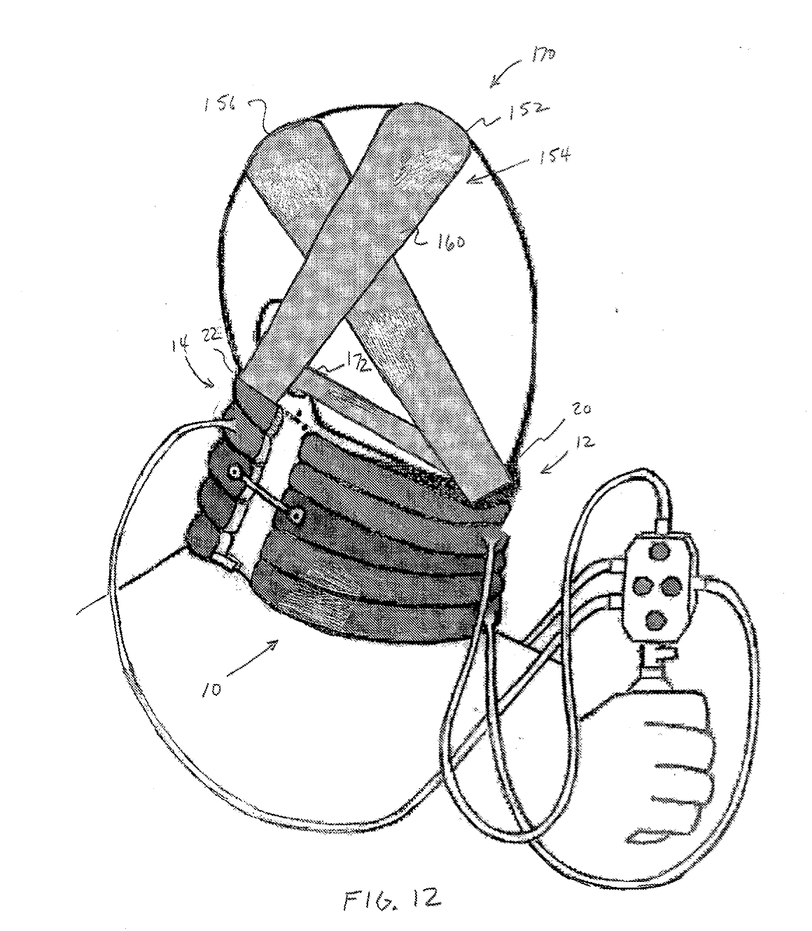

FIG. 12 is an isometric view of a head harness system 170, according to the various embodiments. The head harness system 170 may include the forehead strap 152 may also include the crown strap 156, as previously described. The head harness system 170 may also include a coupling strap 172 that may extend between the forehead strap 152 and the crown strap 156. The coupling strap 172 may be formed from any flexible and resilient material, including various fabrics and polymers having elastomeric properties. In other embodiments, the coupling strap 172 may be formed from any flexible and resilient material, including various fabrics and polymers having approximately non-elastic properties. The coupling strap 172 may be coupled to the forehead strap 152 and the crown strap 156 using adhesives, or using a variety of mechanical fasteners, such as releasable snaps, buttons, rivets, or even hook and loop fasteners, or by welding. The coupling strap 172 may enhance the effectiveness of the head harness system 170 by preventing movement of the front portion 12 away from the rear portion 14 as the various chambers (e.g., the front bottom chamber 18, the rear bottom chamber 26, the front top chambers 20, the left rear top chamber 24 and the right rear top chamber 22) are extended.

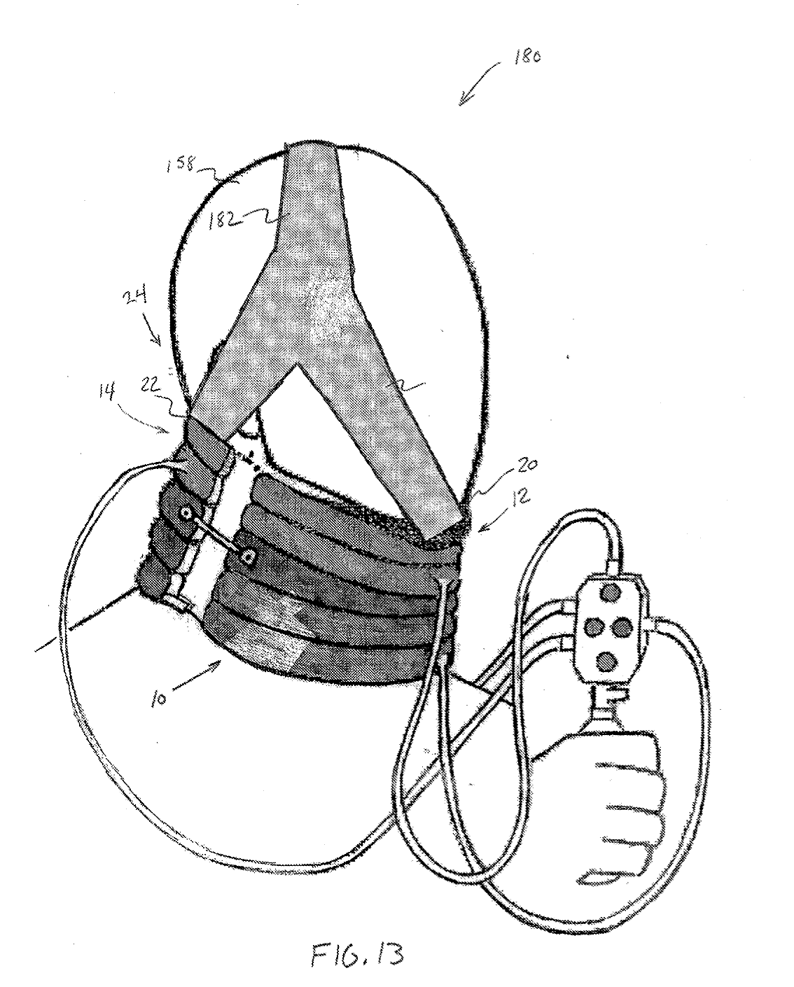

FIG. 13 is an isometric view of a head harness system 180, according to the various embodiments. The head harness system 180 may include a head strap 182 that may be coupled to selected locations on the rear portion 14 and extend across the crown 158 of the user. The head strap 182 may also be coupled to selected locations on the front portion 20. For example, the head strap 182 may be coupled to the right rear top chamber 22 of the multi-directional cervical traction collar 10 at a first end, and to the left rear top chamber 24 at an opposing second end. The head strap 182 may also be coupled to the one or more top front chambers 20. The head strap 182 may be formed from any flexible and resilient unitary material, including various fabrics and polymers having elastomeric properties. Alternatively, the head strap 182 may be formed from a plurality of separate elements that may be joined to form a unitary assembly using adhesives, mechanical fasteners, or welding.

In other embodiments, the various forehead straps 152 and crown straps 156 may be constructed of a flexible but non-elastic material, in which case the length of the strap is determined by adjusting one or more releasable ends of the strap through a fastening method such as, but not limited to, loop and hook fasteners.

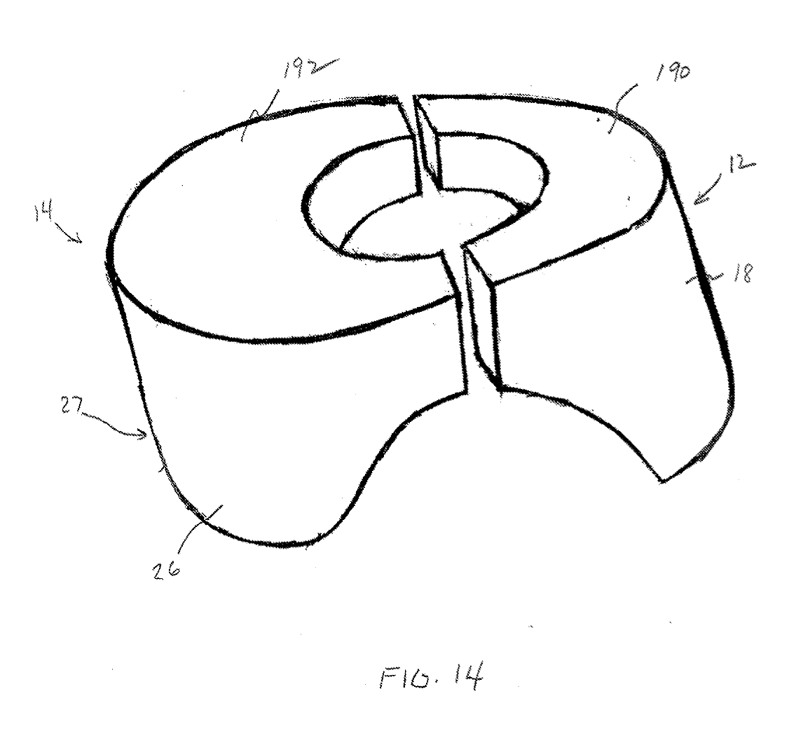

FIG. 14 is an isometric side view of the front bottom chamber 18 of the front portion 12 of the collar 10, and of the rear bottom chamber 26 of the rear portion 14 of the collar 10. As described above, the front bottom chamber 18 of the front portion 12, and the rear bottom chamber 26 of the rear portion 14 may join at left and right sides of the neck of the user to surround and enclose the neck of the user. The front portion 12 may include a top surface 190 that may serve as a base for the one or more top front chambers 20. The rear portion 14 may also include a top surface 192. The top surface 192 may serve as a base for one or more of the right rear top chamber 22 and the one or more of left rear top chamber 24. In accordance with the various embodiments, the front bottom chamber 18 of the front portion 12 may not be present, so that the top surface 190 may abut, or be at least proximate to chest and shoulder portions of the user. Similarly, the rear portion 14 may not include the rear bottom chamber 26, so that the top surface 192 may abut, or be at least proximate to back and shoulder portions of the user. The top surfaces 190 and 192 may be generally oriented transversely to a longitudinal axis of the neck and may be suitably dimensioned to provide a stable base for the top front chambers 20 and the one or more of the right rear top chamber 22 and the one or more of the left rear top chamber 24.

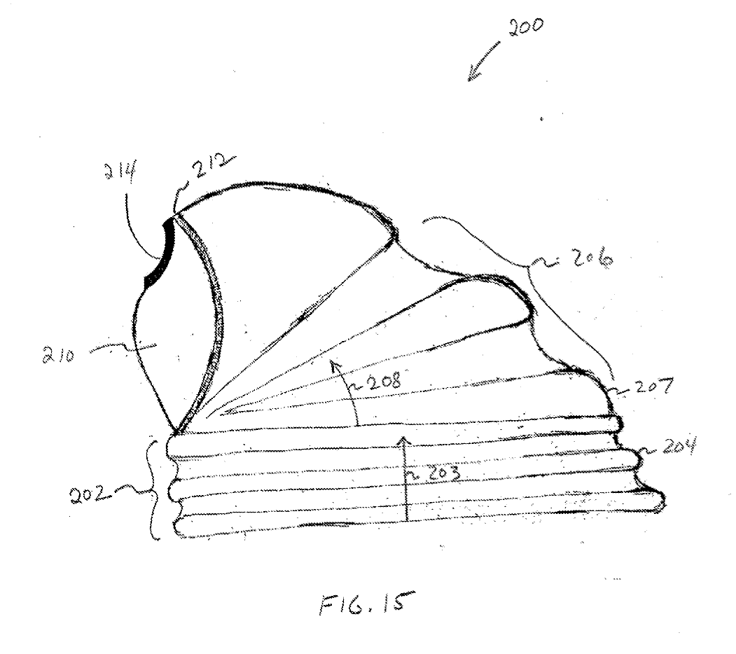

FIG. 15 is a side elevational view of a rear chamber 200, according to the various embodiments. Although FIGS. 1-3 may show embodiments with two top rear chambers (e.g., the right rear top chamber 22 and the left rear top chamber 24), in other embodiments, the right rear top chamber 22 and the left rear top chamber 24 may be further sub-divided into a multiplicity of separately inflatable chambers. Alternately, other embodiments have a single top rear chamber. The rear chamber 200 may include a first stage 202 that may be configured to extend in a first, generally cephalic, direction 203. The first stage 202 may include a bellows assembly that further includes a plurality of pleated sections 204 to permit extension in the first direction when the first stage 202 is internally pressurized. The rear chamber 200 may also include a second stage 206 that may also include pleated section 207 that permit the second stage 206 to extend in a second, generally anterior, direction 208 as the second stage 206 is extended. A third stage 210 may be included in the rear chamber 200 that is configured to extend and conform to a shape of the occiput of the user. Accordingly, the third stage 210 may be configured as a relatively shape-conforming bladder. The third stage 210 may also include a relatively flexible rim 212 that may couple to one or more of the forehead strap 152 of FIG. 11, or the head strap 182 of FIG. 13. A fourth stage 214 may be included in the rear chamber 200, made of a rigid or semi-rigid material that may provide a firmer contact with the occipital region of the user.

Still referring to FIG. 15, it is understood that the first stage 202 of the rear chamber 200 may be positioned on the top surface 192 (FIG. 14) of the rear portion 14. In embodiments that do not have the rear bottom chamber 26, the first stage 202 may rest on the posterior aspect of the top of the shoulders, which may overlay the trapezius muscle and the upper back of the user. In some embodiments, the pleated sections 204 may be replaced with a bladder that includes a concave downward membrane configured to extend into a convex downward shape in order to generate a caudal, or downward, push on the top of the shoulders and an upward, or cephalic lift on the back of the head of the user. In the various embodiments, it is also understood that a material used in parts of the second stage 206 may vary in thickness and rigidity from a material used in other parts of the second stage 206. For example, in some embodiments, a decreased material rigidity may be useful in some areas because it may permit the second stage 206 to generate increased lift against the back of the head. An increased rigidity, in contrast, may be useful in other areas because it may resist posterior displacement of the second stage 206 during extension. The relatively wide anterior-posterior depth of the second stage 206 may also be structured to encapsulate a deep column of air that may add rigidity and also resist posterior displacement of the second stage 206 during extension. The third stage 210 may be continuously adjoin the second stage 206, and may consist of a thin, resilient elastic membrane that may retract into a concave anterior crevice of the second stage 206 when the third stage 210 is retracted, and may extend to a generally convex anterior shape that conforms to the contour of the back of the head for improved conformity and lifting force. As previously mentioned, some embodiments may also include the fourth stage 214, which may include an occipital cup that may attach to the elastic membrane, and which may contact the occipital region of the user. The occipital cup may be made of a rigid or semi rigid material, or even a semi rigid gel, although other materials may also be used. In some embodiments, the occipital cup may be fixedly attached to the flexible membrane, while in other embodiments the occipital cup may be removably coupled to the flexible membrane by a releasable means such as a hook and loop fastener. The detachable occipital cup may permit the user to select remove the occipital cup, if desired. Accordingly, the flexible membrane may contact the head of the user directly. Alternatively, the user may select an occipital cup from a standardized set or customized occipital cup that may be configured to fit a shape of an occipital bone of the user. In some embodiments, the second strapping mechanism 17 (FIG. 1) may run continuously between the one or more top front chambers 20 and an underside of the chin cup 21 (FIG. 1). In other embodiments, the second strapping mechanism 17 may be coupled to one or more of the lateral aspects of the chin cup 21 and the most lateral aspect of the one or more top front chambers 20.

While the various embodiments have been illustrated and described, as noted above, many changes can be made without significantly departing from the spirit and scope of the disclosed embodiments. Accordingly, the scope is not necessarily limited by the disclosure of the foregoing embodiments.

I claim:

1. An apparatus, comprising:
a cervical collar configured to be positioned on a neck of a user and including a front portion having at least one extensible chamber and an opposing rear portion having at least one extensible chamber, wherein the front section and the rear section are mutually removably coupleable.

2. The apparatus of claim 1, comprising a strapping mechanism extending between the front portion and the rear portion that is configured to adjustably couple the front portion to the rear portion.

3. The apparatus of claim 2, comprising one or more extensible top front chambers in the front portion, and one or more extensible top rear chambers in the rear portion, wherein the strapping mechanism comprises a first strapping mechanism configured to couple the top front chambers and the top rear chambers.

4. The apparatus of claim 2, comprising at least one extensible bottom chamber in the front portion, and one or more extensible rear bottom chambers in the rear portion, wherein the strapping mechanism comprises a second strapping mechanism configured to couple the front bottom chamber and the rear bottom chambers.

5. The apparatus of claim 2, comprising at least one side opening between the first portion and the second portion, wherein the strapping mechanism is configured to extend across the at least one side opening.

6. The apparatus of claim 1, wherein the one or more extensible chambers of the front portion, and the one or more extensible chambers of the rear portion are configured to impart at least one non-axial force on a head of the user.

7. The apparatus of claim 1, comprising a controller coupleable to a source of a pressurized fluid that is fluidly coupled to the at least one extensible chamber of the front section and the at least one extensible chamber of the rear section.

8. An apparatus, comprising:
a cervical collar configured to be positioned on a neck of a user and including a front portion having at least one extensible chamber and an opposing rear portion having at least one extensible chamber, wherein the front section and the rear section are mutually removably coupleable by at least one strapping mechanism extending between the front portion and the rear portion; and
a head harness system coupled to the front portion and the rear portion configured to extend across a head of the user.

9. The apparatus of claim 8, wherein the head harness system comprises a forehead strap coupled to selected locations on the rear portion and configured to extend across a forehead of the user.

10. The apparatus of claim 8, wherein the head harness system comprises a crown strap coupled to selected locations on the front portion and configured to extend across a crown of the user.

11. The apparatus of claim 8, wherein the head harness system comprises a forehead strap coupled to selected locations on the rear portion and configured to extend across a forehead of the user, and a crown strap coupled to selected locations on the front portion and configured to extend across a crown of the user, further wherein the head harness comprises a coupling strap extending between the forehead strap and the crown strap.

12. The apparatus of claim 8, wherein the head harness system comprises a head strap coupled to selected locations on the front portion and the rear portion, further wherein the head strap extends at least across a crown of the user.

13. The apparatus of claim 8, comprising at least one strapping mechanism extending between the front portion and the rear portion that is configured to adjustably couple the front portion to the rear portion.

14. The apparatus of claim 13, wherein the strapping mechanism extends across at least one side opening in the collar.

15. A method, comprising:
positioning a cervical collar about a neck of a user;
positioning a mouth piece in the user that is configured to engage the dentition of the user;
extending selected chambers in the cervical collar to urge the collar into contact with at least one of a chin and an occipital region of the user; and
tractioning the neck of the user using a selected mobilization pattern.

16. The method of claim 15, wherein positioning the cervical collar comprises adjusting at least one strapping mechanism to retain the cervical collar on the user.

17. The method of claim 15, wherein positioning a mouth piece comprises positioning the mouth piece on a lower mandible and proximate to a temporomandibular joint of the user.

18. The method of claim 15, wherein extending selected chambers in the cervical collar comprises extending one or more bottom chambers and top chambers in the collar.

19. The method of claim 16, comprising forward chambers and rear chambers opposite the forward chambers, wherein tractioning the neck comprises:
extension of the rear chambers to mobilize the neck in forward flexion;
extension of the front chambers to mobilize the neck in backwards extension;
extension of a selected rear chamber to mobilize the neck in a first lateral flexion;
extension of a selected front chamber and a selected rear chamber to mobilize the neck in a first lateral flexion;
extension of a rear chamber other than the selected rear chamber to mobilize the neck in a second lateral flexion; and
extension of a front chamber other than the selected front chamber and extension of a rear chamber other than the selected rear chamber to mobilize the neck in a second lateral flexion.

20. The method of claim 15, comprising removing the cervical collar from the user by retracting the selected chambers.
